(19) United States
(12) Patent Application Publication
(10) Pub. No.: US 2016/0058427 A1
(43) Pub. Date: Mar. 3, 2016

(54) ULTRASOUND PROBE AND ULTRASOUND DIAGNOSTIC APPARATUS

(71) Applicant: Konica Minolta, Inc., Tokyo (JP)

(72) Inventors: Morio NISHIGAKI, Fujisawa-shi (JP); Yoshihiro TAKEDA, Tokyo (JP)

(21) Appl. No.: 14/824,659

(22) Filed: Aug. 12, 2015

(30) Foreign Application Priority Data
Aug. 28, 2014 (JP) 2014-173730

(51) Int. Cl. A61B 8/00; A61B 8/08; A61B 8/14

(52) U.S. Cl. CPC A61B 8/54; A61B 8/4494; A61B 8/145; A61B 8/4461; A61B 8/467; A61B 8/5207; A61B 8/5215; A61B 8/463

(57) ABSTRACT

An ultrasound probe and an ultrasound diagnostic apparatus are disclosed. According to one implementation, an ultrasound probe includes the following. A plurality of transducers are arrayed in at least a predetermined first direction and transmit ultrasound to a subject to receive a reflected wave. A convergence unit converges the ultrasound transmitted and received by the plurality of transducers to the first direction. A switching element turns off transmission and reception of the ultrasound of some of the plurality of transducers arrayed in the first direction. A direction switching setter performs switching operation of turning on and turning off of the switching element to deflect in the first direction a transmission/reception direction of the ultrasound by the plurality of transducers.

### BACKGROUND OF THE INVENTION

1. Field of the Invention

The present invention relates to an ultrasound probe and an ultrasound diagnostic apparatus.

2. Description of Related Art

Conventionally, there is an ultrasound diagnostic apparatus in which the ultrasound is irradiated in the subject, the reflected wave (echo) is received and predetermined signal data processing is performed to examine the internal structure of the subject. Such ultrasound diagnostic apparatus is used for various purposes such as examination and treatment for medical purposes or examination inside a building structure.

In addition to processing the data of the obtained reflected wave and displaying the image, the ultrasound diagnostic apparatus is used for punching a puncture needle into a target position while confirming by sight the puncture needle and the target position when used in obtaining a sample of a specific site (target) in the subject, draining water or inserting and leaving medicine or a marker in a specific site. By using such ultrasound image, treatment of the target in the subject can be performed speedily, reliably, and easily.

Most ultrasound diagnostic apparatuses that are used include transducers which transmit and receive ultrasound arrayed in a straight line or a two dimensional matrix. Here, the position and direction where the ultrasound is transmitted and received is scanned in a predetermined array direction (specifically, electronic scanning) to perform imaging. The puncture needle is inserted along the scanning direction so that the puncture needle is positioned within a range where continuous imaging is possible by existence of the ultrasound beam from the position where the puncture needle is inserted in the subject until the puncture needle reaches the target.

However, the puncture needle does not always accurately advance in the first inserting direction or the puncture needle may be bent due to the internal state or structure of the subject or the shape of the tip of the puncture needle. As a result, there is a problem that the tip of the puncture needle falls outside the range where imaging is possible by the existence of the ultrasound beam in a width direction perpendicular to the scanning direction, and imaging is not possible.

When a simple cross-sectional image is obtained, a user who is not used to the apparatus may not be able to suitably change the posture of the ultrasound probe when making fine adjustments of the imaging range in the short axis direction. This causes the problem of obtaining the desired image becoming troublesome.

Japanese Patent Application Laid-Open Publication No. 2000-139926 describes providing a delay circuit which delays the timing of the plurality of transducers arrayed in a width direction and switching the relation of the size of the delay amount of the plurality of transducers to deflect the transmitting and receiving direction of the ultrasound so that imaging outside the original width of transmission and reception of the ultrasound is possible.

However, a deflection control circuit including such delay circuit for changing and adjusting the imaging range is large in size or emits heat in operation. Therefore, the usability of the ultrasound probe reduces.

### BRIEF SUMMARY OF THE INVENTION

The present invention has been made in consideration of the above problems, and one of the main objects is to provide an ultrasound probe and an ultrasound diagnostic apparatus which is able to change and adjust a range of imaging with an easier structure without reducing usability.

In order to achieve at least one of the above-described objects, according to an aspect of the present invention, there is provided an ultrasound probe including:

a plurality of transducers which are arrayed in at least a predetermined first direction and which transmit ultrasound to a subject to receive a reflected wave;

a convergence unit which converges the ultrasound transmitted and received by the plurality of transducers to the first direction;

a switching element which turns off transmission and reception of the ultrasound of some of the plurality of transducers arrayed in the first direction; and

a direction switching setter which performs switching operation of turning on and turning off of the switching element to deflect in the first direction a transmission/reception direction of the ultrasound by the plurality of transducers.

Preferably, in the ultrasound probe, the convergence unit is formed so that focal positions of the ultrasound transmitted and received by the plurality of transducers arrayed in the first direction are set in a plurality of different positions.

Preferably, the ultrasound probe further includes:

a switching controller which outputs a control signal to the direction switching setter,

wherein, the direction switching setter performs the switching operation of the switching element based on the control signal.

According to another aspect of the present invention, there is provided an ultrasound diagnostic apparatus including:

the ultrasound probe;

a transmission/reception processor which controls the ultrasound probe to transmit and receive the ultrasound; and

a switching controller which outputs a control signal to the direction switching setter,

wherein, the direction switching setter performs the switching operation of the switching element based on the control signal.

Preferably, the ultrasound diagnostic apparatus further includes:

an image generator which generates a diagnostic image regarding a structure of a subject in a cross-section including a transmission/reception direction of the ultrasound and a second direction perpendicular to the transmission/reception direction and the first direction based on the received ultrasound; and

a puncture needle identifier which identifies a tip portion of the puncture needle in the diagnostic image when a puncture needle is inserted inside the subject along the cross-section,

wherein, the switching controller outputs the control signal which changes a direction of the deflection when the tip portion is not detected in the diagnostic image; and

the puncture needle identifier detects the tip portion of the puncture needle from the diagnostic image regarding the structure in the cross-section according to the changed direction of deflection.

### BRIEF DESCRIPTION OF THE DRAWINGS

The present invention will become more fully understood from the detailed description given hereinbelow and the appended drawings, and thus are not intended to define the limits of the present invention, and wherein;

FIG. 1 is a diagram showing an entire configuration of an ultrasound diagnostic apparatus of an embodiment of the present invention;

FIG. 2 is a block diagram showing a functional configuration of the ultrasound diagnostic apparatus;

FIG. 3 is a diagram showing an example of a transducer array in an ultrasound probe;

FIG. 4A is a diagram showing a cross-sectional structure along a short axis direction in the transducer array of the ultrasound probe of the first embodiment;

FIG. 4B is a diagram showing another cross-sectional structure along a short axis direction in the transducer array of the ultrasound probe of the first embodiment;

FIG. 5 is a diagram showing a relation between on/off of a switching element and a transmission/reception direction of ultrasound;

FIG. 6 is a diagram showing a result calculating a difference in gain according to the used transducer;

FIG. 7 is a diagram showing an example of on/off control of a transducer in each transducer line in the scanning direction;

FIG. 8A is a diagram showing a state of positioning the ultrasound probe and a puncture needle in the ultrasound diagnostic apparatus of the present embodiment;

FIG. 8B is a diagram showing an example of a position display image of the puncture needle;

FIG. 8C is a diagram showing an example of a position display image of the puncture needle;

FIG. 9A is a diagram showing an example of a position display image of the puncture needle;

FIG. 9B is a diagram showing an example of a position display image of the puncture needle;

FIG. 10 is a flowchart showing a control process of display control processing of a puncture needle performed in the ultrasound diagnostic apparatus;

FIG. 11 is a diagram showing a modification 1 of a cross-sectional structure along a short axis direction regarding the transducer array in the ultrasound probe;

FIG. 12A is a diagram showing a cross-sectional structure along a short axis direction in the transducer array of the ultrasound probe of the second embodiment;

FIG. 12B is a diagram describing a transmission and reception direction of the ultrasound by the transducer;

FIG. 13A is a diagram showing modification 2 of a cross-sectional structure along a short axis direction in the transducer array of the ultrasound probe; and

FIG. 13B is a diagram describing a transmission and reception direction of the ultrasound by the transducer.

### DETAILED DESCRIPTION OF PREFERRED EMBODIMENTS

An embodiment of the present invention is described with reference to the drawings.

### First Embodiment

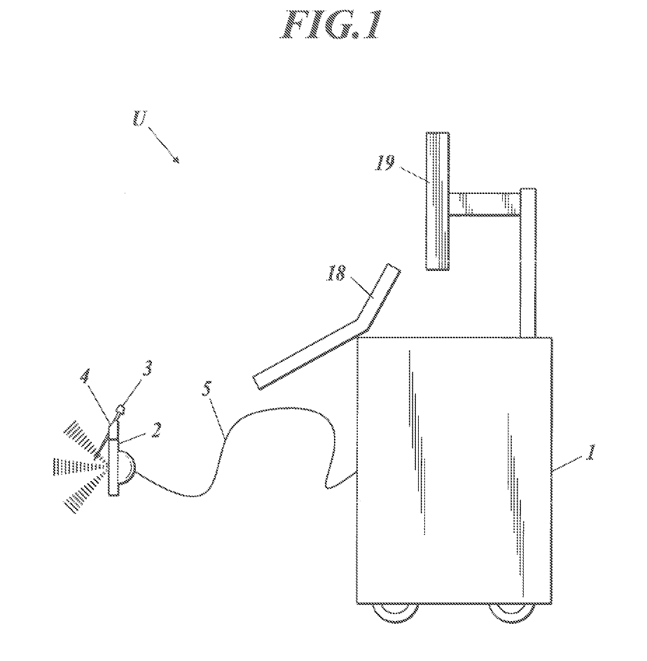

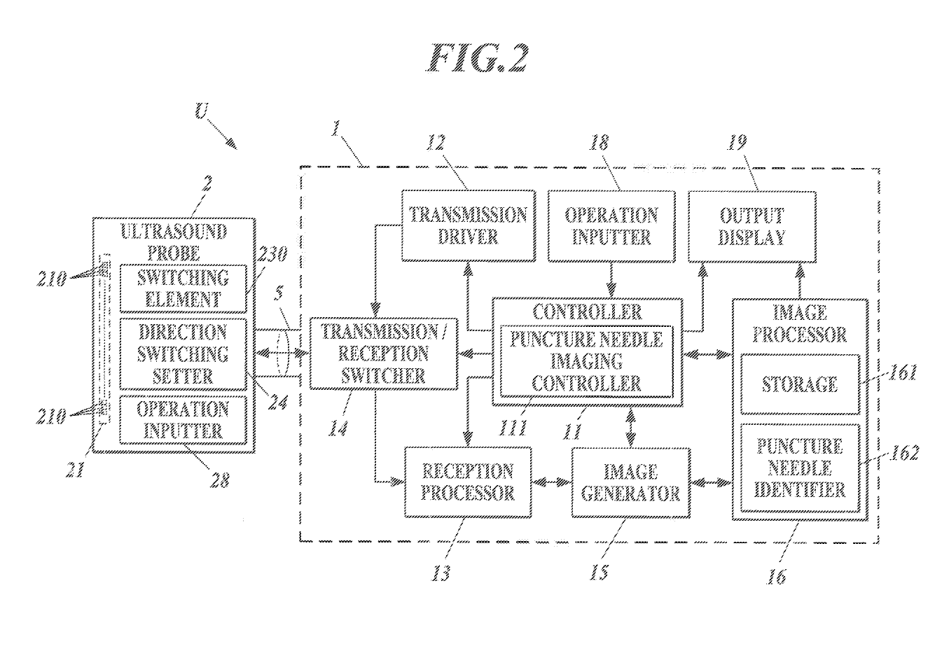

FIG. 1 is an entire diagram of an ultrasound diagnostic apparatus U of the first embodiment. FIG. 2 is a block diagram showing an internal configuration of the ultrasound diagnostic apparatus U.

As shown in FIG. 1, the ultrasound diagnostic apparatus U includes an ultrasound diagnostic apparatus main body 1, an ultrasound probe 2 connected to the ultrasound diagnostic apparatus main body 1 through the cable 5, a puncture needle 3, and an attachment 4 attached to the ultrasound probe 2.

The puncture needle 3 includes a hollow long needle shape and is inserted in the subject in an angle determined by a setting of the attachment 4. The puncture needle 3 can be changed to a needle with a suitable thickness, length and tip form depending on the type or amount of the target (subject) to be collected or the medicine inserted.

The attachment 4 holds the puncture needle 3 in a set direction. The attachment 4 is attached to the side of the ultrasound probe 2, and the direction of the puncture needle 3 can be suitably changed and set depending on the angle that the puncture needle 3 is inserted in the subject. The attachment 4 can simply move the puncture needle 3 in the inserting direction and also insert the puncture needle 3 while spinning around a central axis of the puncture needle 3.

Instead of the attachment 4, a guide which holds the puncture needle 3 in the inserting direction can be provided directly in the ultrasound probe 2.

An operation inputter 18 and an output display 19 (display) are provided in the ultrasound diagnostic apparatus main body 1. As shown in FIG. 2, the ultrasound diagnostic apparatus main body 1 also includes the controller 11 (display controller), a transmission driver 12, a reception processor 13, a transmission/reception switcher 14, an image generator 15, and an image processor 16.

According to input operation from outside to an input device such as a keyboard or a mouse of the operation inputter 18, the controller 11 of the ultrasound diagnostic apparatus main body 1 outputs a driving signal to the ultrasound probe 2 to output ultrasound or obtains a reception signal regarding the reception of the ultrasound from the ultrasound probe 2 to perform various processing, and displays the result on, for example, the display screen of the output display 19 according to necessity.

The controller 11 includes a CPU (Central Processor), a HDD (Hard Disk Drive), and a RAM (Random Access Memory). The CPU reads various programs stored in the HDD to be loaded on the RAM, and centrally controls operation of each section of the ultrasound diagnostic apparatus U according to the program. The HDD stores a control program and various processing programs to operate the ultrasound diagnostic apparatus U and various setting data, etc. Other than the HDD, such programs and setting data can be stored in a rewritable state in, for example, an auxiliary storage apparatus using a nonvolatile memory such as a flash memory including a SSD (Solid State Drive). The RAM is a volatile memory such as a SRAM, DRAM, etc. and provides a memory space for the job to the CPU and temporarily stores data.

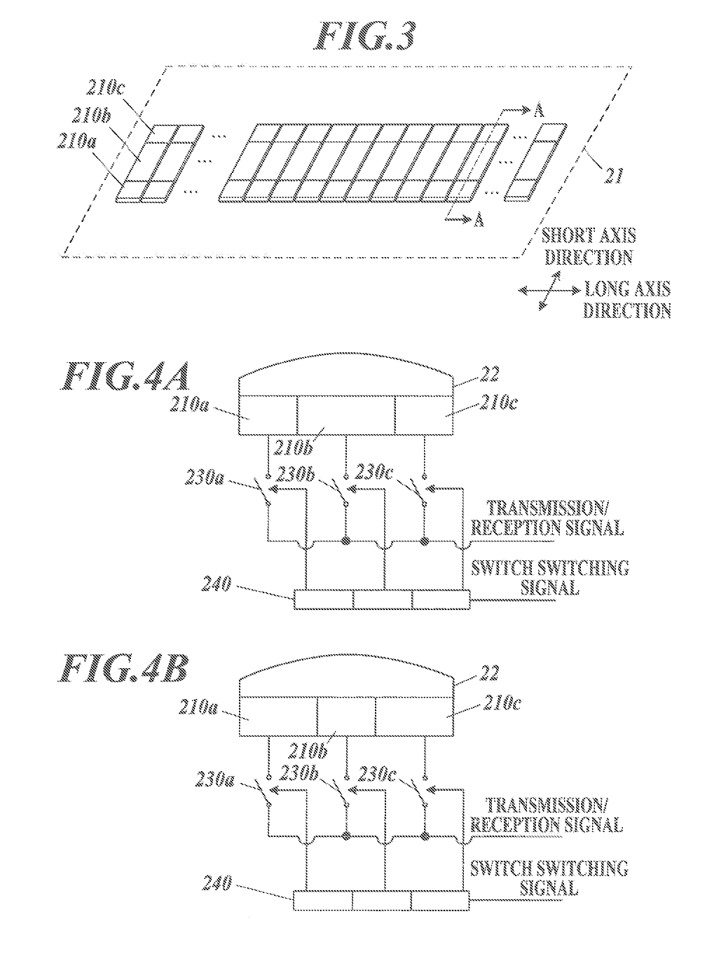

The controller 11 includes a puncture needle imaging controller 111 (switching controller). Based on position information of the puncture needle 3 identified by the image processor 16, when the tip of the puncture needle 3 is deflected to the perpendicular direction with respect to the scanning direction of a transducer array 21 and falls out from an imaging range, the puncture needle imaging controller 111 sets the imaging direction by the transducers 210a to 210c (see FIG. 3) arrayed in the short axis direction to be deflected and outputs the control signal for the above setting. The operation of the puncture needle imaging controller 111 can be performed by software using the CPU or the RAM of the controller 11.

The transmission driver 12 outputs a pulse signal provided to the ultrasound probe 2 according to the control signal input from the controller 11 so that the ultrasound probe 2 emits ultrasound. For example, the transmission driver 12 includes a clock generating circuit, a pulse width setter, a pulse generating circuit and a delay circuit. The clock generating circuit is a circuit which generates a clock signal to determine transmission timing and transmission frequency of the pulse signal. The pulse width setter sets the waveform (shape), voltage amplitude, and pulse width of the transmission pulse output from the pulse generating circuit. The pulse generating circuit generates the transmission pulse based on the setting of the pulse width setter and outputs the above to a different line path for each transducer 210 of the ultrasound probe 2. The delay circuit counts the clock signal output from the clock generating circuit. When the set delay time passes, the pulse generating circuit generates the transmission pulse to be output to the line path.

The reception processor 13 is a circuit which obtains the reception signal input from the ultrasound probe 2 according to control from the controller 11. For example, the reception processor 13 is provided with an amplifier, an A/D conversion circuit, and a phasing adding circuit. The amplifier is a circuit which amplifies the reception signal according to the ultrasound received by each transducer 210 of the ultrasound probe 2 at a predetermined amplifying percentage set in advance. The A/D conversion circuit is a circuit which converts the amplified reception signal to digital data at a predetermined sampling frequency. The phasing adding circuit is a circuit which provides a delay time to the A/D converted reception signal for each line path corresponding to each transducer 210, adjusts the time phase, and adds (phasing adding) the above to generate sound ray data.

Based on control of the controller 11, the transmission/reception switcher 14 switches between transmitting a driving signal from the transmission driver 12 to the transducer 210 when the ultrasound is emitted (transmitted) from the transducer 210, and outputting a reception signal to the reception processor 13 when the signal regarding the ultrasound output by the transducer 210 is obtained.

The transmission/reception processor is composed of the transmission driver 12, the reception processor 13, and the transmission/reception switcher 14.

The image generator 15 generates the diagnostic image based on the ultrasound reception data. The image generator 15 detects (envelope detection) the sound ray data input from the reception processor 13 to obtain the signal, and performs logarithmic amplifying, filtering (for example, low pass filtering, smoothing), enhancement processing, etc. as necessary. The image generator 15 generates frame image (diagnostic image) data of B mode-display showing two dimensional structure in a cross-section including a transmitting direction (depth direction of subject) of the signal with the brightness signal according to the strength of the signal and a scanning direction (second direction) of the ultrasound transmitted by the ultrasound probe 2. Here, the image generator 15 is able to adjust dynamic range and perform gamma correction regarding the display. The image generator 15 can include a CPU or a RAM dedicated to be used for generating the above images. Alternatively, the image generator 15 may be provided by forming a hardware configuration dedicated to generating the image on a substrate (ASIC (Application Specific Integrated Circuit), etc.) or by forming with a FPGA (Field Programmable Gate Array). Alternatively, the image generator 15 can be a configuration which performs processing regarding generating the image with a CPU and a RAM of the controller 11.

The image processor 16 includes a storage 161 and a puncture needle identifier 162.

The storage 161 stores diagnostic image data (frame image data) used for real time display after being processed by the image generator 15 or display similar to the above. The diagnostic image data is stored in a unit of frames in the amount of a predetermined number of the latest frames. The storage 161 is a volatile memory such as a DRAM (Dynamic Random Access Memory). Alternatively, the storage 161 can be various types of nonvolatile memory which is rewritable with high speed. The diagnostic image data stored in the storage 161 is read according to control of the controller 11, transmitted to the output display 19, and output outside the ultrasound diagnostic apparatus U through the communicator which is not shown. Here, when the display method of the output display 19 is a television type, a DSC (Digital Signal Converter) is provided between the storage 161 and the output display 19, and the data is output after converting the scanning format.

The puncture needle identifier 162 generates image data to identify the position of the puncture needle 3, and performs suitable processing on the image data to identify the position of the tip portion of the puncture needle 3. The tip portion identified here is not limited to the tip of the puncture needle 3, and can be a predetermined range near the tip of the puncture needle 3 according to the accuracy of detection, image resolution, etc. The punctured needle identifier 162 can be a shared CPU and RAM of the image processor 16 or can be provided with a dedicated CPU and RAM. Alternatively, the punctured needle identifier 162 can be realized by various processing performed by the CPU and the RAM of the controller 11. The punctured needle identifier 162 can store tip position information of the identified punctured needle 3 as history.

The method of identifying the position of the puncture needle 3 is described below. For example, the difference or correlation between a plurality of diagnostic images generated with predetermined intervals of time can be obtained to detect a moving tip 3a (tip portion) of the puncture needle 3. Alternatively, the position of the tip 3a can be predicted based on movement history of the tip 3a, and the tip 3a can be detected based on the predicted position. Further, the outline can be detected and the user can select from the candidates obtained first by input on the operation inputter 18, and the outline similar to the selected outline can be detected based on the predicted position.

The operation inputter 18 includes a press button switch, a keyboard, a mouse, a trackball or a combination of the above. The operation inputter 18 converts the input operation of the user to an operation signal and inputs the signal to the ultrasound diagnostic apparatus main body 1.

The output display 19 includes a display screen employing any one of various types of display methods such as a LCD (Liquid Crystal Display), an organic EL (Electro-Luminescent) display, a non-organic EL display, a plasma display, a CRT (Cathode Ray Tube) display and a driver for the above. The output display 19 generates a control signal output from the CPU 15, and a driving signal of the display screen (each display pixel) according to the image data generated in the image processor 16. With this, a menu, or status regarding the ultrasound diagnosis or measurement data based on the received ultrasound is displayed on the display screen. The output display 19 can be provided with a separate LED lamp and display whether the power is turned on or off.

Such operation inputter 18 and output display 19 can be provided together with the case of the ultrasound diagnostic apparatus main body 1 or attached externally through a RGB cable, a USB cable, a HDMI (registered trademark) cable or the like. When the ultrasound diagnostic apparatus main body 1 is provided with an operation input terminal or a display output terminal, conventional peripheral equipment for operation and display can be connected to such terminals and used.

The ultrasound probe 2 oscillates ultrasound (here, about 1 to 30 MHz) and emits the ultrasound to a subject such as a live body and also functions as an acoustic sensor which receives the reflected wave (echo) reflected on the subject among the emitted ultrasound to convert the reflected wave to an electric signal. The ultrasound probe 2 includes a transducer array 21 which is an array of a plurality of transducers 210 which transmit and receive ultrasound, a plurality of switching elements 230 each corresponding to the transducer 210, a direction switching setter 24, and an operation inputter 28 (inputter).

Here, the ultrasound probe 2 emits ultrasound from outside (surface) to the inside of the subject and the reflected wave is received. The ultrasound probe 2 includes those with a size and shape to be used inside a digestive tract or blood vessel, or to be used inserted in a body cavity.

The user places the transmission/reception face of the ultrasound in the ultrasound probe 2, in other words, the face in the direction that the ultrasound is emitted from the transducer array 21 in contact with the subject with a predetermined pressure, operates the ultrasound diagnostic apparatus U, and performs ultrasound diagnosis.

The transducer array 21 is an array of a plurality of transducers 210 provided with a piezoelectric element including a piezoelectric body and an electrode provided at both ends where charge appears due to deformation (expand and contract) of the piezoelectric body.

FIG. 3 is a diagram showing an example of a transducer array 21 in an ultrasound probe 2 of the present embodiment.

According to the ultrasound diagnostic apparatus U of the present embodiment, the transducer array 21 includes a plurality of transducers 210 arranged in a matrix shape in a two-dimensional plane (does not have to be flat) defined by a predetermined direction (scanning direction, second direction), and a width direction orthogonal to the scanning direction (first direction). Usually, the number of array of the transducer 210 in the scanning direction is larger than the number of array of the transducer 210 in the width direction. Therefore, the scanning direction becomes the long axis direction and the width direction becomes the short axis direction. Here, 3 transducers 210a, 210b, and 210c are positioned in order in the short axis direction. The group of 3 transducers 210a to 210c in the short axis direction is also described as a transducer line (array) below.

A voltage pulse is supplied to the plurality of transducers 210 in a unit of a predetermined number of transducer lines in the scanning direction in order (includes some duplication). With this, the piezoelectric body of the transducer 210 supplied with the voltage pulse deforms (expand and contract) according to an electric field generated in the piezoelectric body and the ultrasound is oscillated. The oscillated ultrasound is emitted to the position and direction according to the position of the transducer 210 included in the predetermined number of transducer lines supplied with the voltage pulse, direction, convergence direction of the oscillated ultrasound and size of the difference (delay) in timing. When ultrasound with a predetermined frequency band enters the transducer 210, the thickness of the piezoelectric body changes (vibrates) by the acoustic pressure. With this, charge according to the amount of change is generated, and the ultrasound is converted to the electric signal according to the charge amount to be output.

The direction switching setter 24 stores the setting to switch (deflect) the transmission/reception direction of the ultrasound to the short axis direction and performs switching operation of turning on and turning off of the switching element 230 corresponding to the transducers 210 according to the setting. According to the ultrasound diagnostic apparatus U of the present embodiment, as described later, the transmission/reception direction of the ultrasound can be set for each transducer line.

The operation inputter 28 receives input from the user and operation according to the content is performed. For example, the setting of the direction switching setter 24 can be changed according to manual operation by the user on the operation inputter 28. When the ultrasound probe 2 is electrically connected to the attachment 4, the inserting operation of the puncture needle 3 can be controlled according to the operation on the operation inputter 28.

The cable 5 includes a connector (not shown) to connect with the ultrasound diagnostic apparatus main body 1 and a connector (not shown) to connect with the ultrasound probe 2 at each end. The ultrasound 2 is connected detachably to the ultrasound diagnostic apparatus main body 1 by the cable 5. The cable 5 can be formed as one with the ultrasound probe 2.

Next, the configuration regarding the deflection of the ultrasound in the transmission/reception direction in the ultrasound diagnostic apparatus U of the present embodiment is described.

FIG. 4A and FIG. 4B are diagrams showing a cross-sectional configuration along the short axis direction in the transducer array 21 of the ultrasound probe 2 of the present embodiment. Here, the cross-sectional configuration at cross-section AA in FIG. 3 is shown.

As shown in FIG. 4A and FIG. 4B, a convex shaped acoustic lens 22 (convergence unit) common to all 3 transducers 210a, 210b, and 210c, arrayed in the short axis direction of the transducer line is provided in the ultrasound probe 2. The ultrasound transmission/reception direction of the transducers 210a, 210b, and 210c is refracted and the transmission/reception width is converged in the short axis direction.

Usually, silicon is used in the acoustic lens 22. Alternatively, other material can be suitably selected according to the desired ultrasound refractive index.

The transducers 210a, 210b, and 210c are connected to the direction switching setter 24 through the switching elements 230a to 230c, respectively. The direction switching setter 24 includes a register 240. According to the switch switching signal stored in the register 240 input in advance from the controller 11, the on and off of the switching elements 230a to 230c is switched and it is determined whether the ultrasound can be received by the transducer line regarding the transducers 210a to 210c. Here, the switching switch signal is transmitted serially and input in the register 240. The operation of the switching elements 230a to 230c can be controlled in parallel so that the number of signal lines between the controller 11 and the register 240 can be reduced.

The switching elements 230a to 230c are not limited. Considering the amount of energy consumed and the pressure resistance performance of the ultrasound reception, for example, FET (field effect transistor) is preferably used. By changing the combination of the switching elements 230a to 230c that are turned off, the transmission/reception direction of the ultrasound by the transducer line is deflected in the short axis direction.

FIG. 5 is a diagram showing a relation between the on and off of the switching elements 230a to 230c and the transmission/reception direction of the ultrasound. Here, the transmission/reception direction of the ultrasound according to the transducers 210a to 210c of FIG. 4A and FIG. 4B is shown with a width of −3 dB (broken line position) in relation to a strength peak value (solid line position) on a sound axis. On the left side of FIG. 5, the position of the transducers 210a to 210c are shown with the marks A to C in the box shown corresponding to the position in the short axis direction.

When the switching element 230a is turned off and the switching elements 230b and 230c are turned on, the driving signal regarding the ultrasound transmission is not transmitted to the transducer 210a, and the reception data regarding the ultrasound reception is not output from the transducer 210a. As a result, the wave surface of the ultrasound transmitted from the transducers 210b and 210c, and received by the transducers 210b and 210c is leaned to the side of the transducer 210a which is not used in the ultrasound transmission and reception.

When the switching element 230b is turned off and only either the switching element 230a or 230c is turned on, the leaning (deflection angle) becomes even larger.

The angle of the leaning is determined by the width of the 3 transducers 210a, 210b, and 210c in the short axis direction. In the example in which the width of the transducers 210a and 210c of both edges is smaller compared to the transducer 210b of the center (FIG. 4A, for example, the ratio between the width of the transducer in the center and the width of the transducers at both edges is 1:1), the deflection angle (angle θa of FIG. 5) when either of the transducers 210a or 210c is turned off (thin line of FIG. 5) becomes smaller than the deflection angle (angle θb of FIG. 5) when either of the transducers 210a or 210c is turned off (thick line of FIG. 5) in an example in which the width of the transducers 210a and 210c of both edges is larger compared to the transducer 210b of the center (FIG. 4B, for example, the ratio between the width of the transducer in the center and the width of the transducer at both edges is 1:2).

In this case, the range of reception expanded to the side deflected when the transmission/reception direction of the ultrasound is deflected changes according to the depth from the ultrasound transmission/reception surface. Specifically, it is difficult to move the imaging range with the operation in the ultrasound diagnostic apparatus U of the present embodiment in the range from the ultrasound transmission/reception surface to about the focal distance of the acoustic lens 22. When the user desires to move the imaging range, the depth of the imaging target (distance from the ultrasound transmission/reception surface) is obtained. If the position is not longer than a predetermined distance determined based on a focal distance of the acoustic lens 22, other methods can be used. For example, both transducers 210a and 210c at both edges can be turned off to make the opened width of the transmission/reception ultrasound smaller to suppress the convergence of the ultrasound so that the imaging range becomes relatively larger. According to such method, an additional configuration does not need to be added to the ultrasound diagnostic apparatus U of the present embodiment, and the method can be used in the processing similar to processing of deflecting the transmission/reception direction of the ultrasound.

Here, since some of the transducers 210a to 210c are not used in transmission and reception, the transmission/reception strength of the ultrasound decreases compared to when all of the transducers 210a to 210c are used. When the generated diagnostic image is displayed on the output display 19 in the ultrasound diagnostic apparatus U, no problems occur for the purpose of confirming by sight the tip of the puncture needle 3 even if the image remains displayed. However, it is easier to view if the brightness distribution (gain) is even (normalized). Therefore, according to the ultrasound diagnostic apparatus U, when the transmission/reception strength of the ultrasound is reduced by not using some of the transducers 210a to 210c, a coefficient in the amount of reduction can be multiplied to the reception strength and the above can be output.

Alternatively, the voltage amplitude of the rectangular wave pulse regarding the transmission of the ultrasound can be changed (raised) and the reception strength can be raised.

When only one of the transducers 210a to 210c is used for transmission and reception, the S/N ratio drastically decreases with the decrease of the ultrasound transmission/reception strength. Therefore, it is necessary to set the width of the transducers 210a to 210c and the voltage amplitude so that the S/N ratio (reception strength) is a value in which the puncture needle 3 is reliably detected.

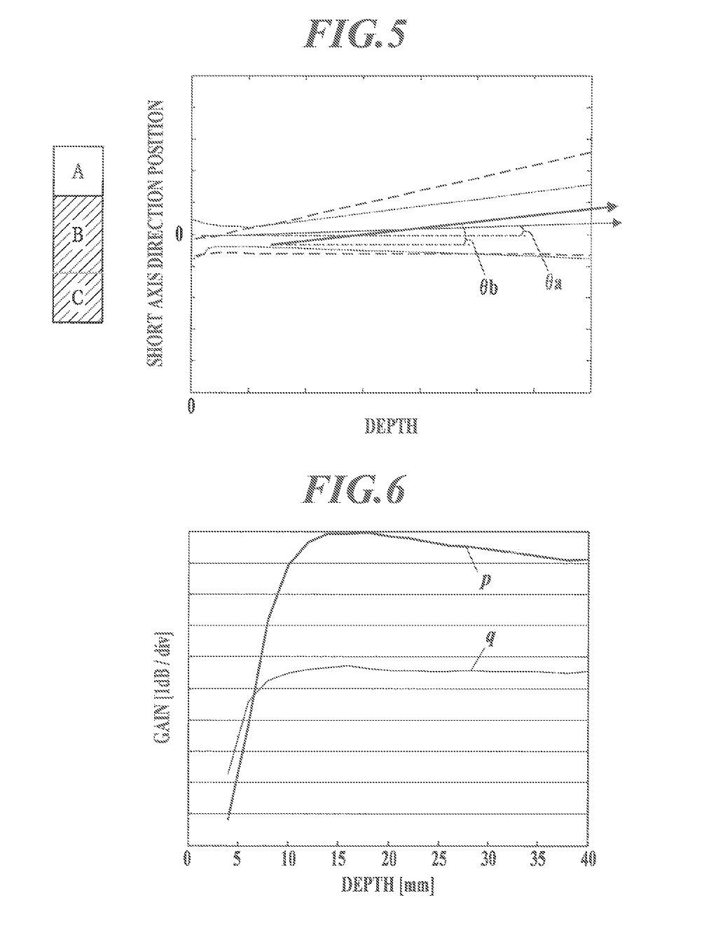

FIG. 6 shows the result of the calculated difference of the gain according to the transducers 210a to 210c which are used.

Here, the gain (p) when all of the transducers 210a to 210c are used and the gain (q) when only the transducer 210b is used is calculated in relation to the distance from the transmission/reception surface of the ultrasound and the result is shown.

As shown in the drawing, the difference of the gain according to the transducer used changes according to the distance from the ultrasound transmission/reception surface due to the difference in the convergence rate of the transmission/reception ultrasound. Therefore, according to the ultrasound diagnostic apparatus U of the present embodiment, the value of the difference of the gain according to the distance is stored in advance in the storage of the controller 11. When any of the transducers 210a to 210c is not used, the coefficient according to the distance from the transmission/reception surface is read, and the value of the obtained reception strength is corrected. With this, normalization is possible with the accurate brightness ratio.

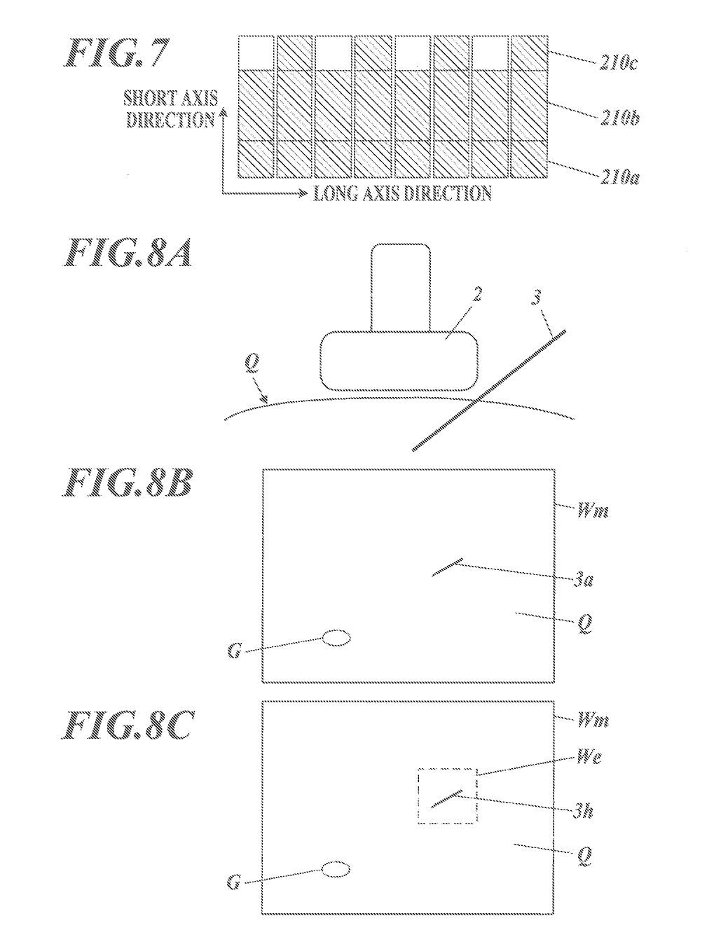

FIG. 7 is a diagram showing an example of on and off control of the transducers 210a to 210c in each line in the scanning direction.

According to the ultrasound diagnostic apparatus U of the present embodiment, in addition to the selection of on and off of the transducers 210a to 210c in the short axis direction, the setting of on and off of the transducers 210a to 210c in the long axis direction can be different for each transducer line.

As one example, described here is an example in which 8 lines of transducer lines are used in one ultrasound transmission and reception along the long axis direction (scanning direction). The transducer line in which the transducer 210c is turned on and the transducer line in which the transducer 210c is turned off is defined alternately and the transmission and the reception of the ultrasound is performed. With this, the central direction of the reception of ultrasound in the short axis direction becomes the middle between the direction of transmission when the transducer 210 is turned on and the direction of transmission when the transducer 210c is turned off. Moreover, the ratio of the combination between the transducer line in which the transducer 210c is turned on and the transducer line in which the transducer 210c is turned off can be changed, and adjustment to further finely change the transmission/reception direction of the ultrasound can be performed.

Here, when the ultrasound is transmitted and received with different transducer lines in different directions at the same time, the reception range of the ultrasound increases but the S/N ratio decreases at both edges. When the inserting direction of the puncture needle 3 becomes away from the target direction (direction approaching the target), it is preferable to finely adjust the central direction of the ultrasound transmission and reception according to the S/N ratio distribution in order to include both the puncture needle 3 and the target in the diagnostic image.

When the ultrasound is transmitted and received with different transducer lines in different directions as described above, if the number of transducer lines which perform transmission and reception of the ultrasound in one direction is small, the profile of the reception strength regarding the transmission and reception of the ultrasound, specifically, the shape of the side lobe tends to be deformed and it may be difficult to obtain the strength distribution accurately due to receiving unnecessary noise. Therefore, in this case, preferable diagnostic images can be generated by setting the number of transducer lines which perform transmission and reception of the ultrasound at once to be larger.

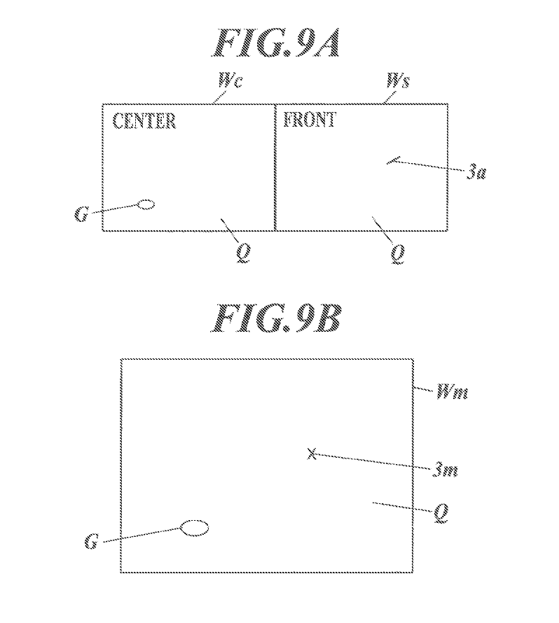

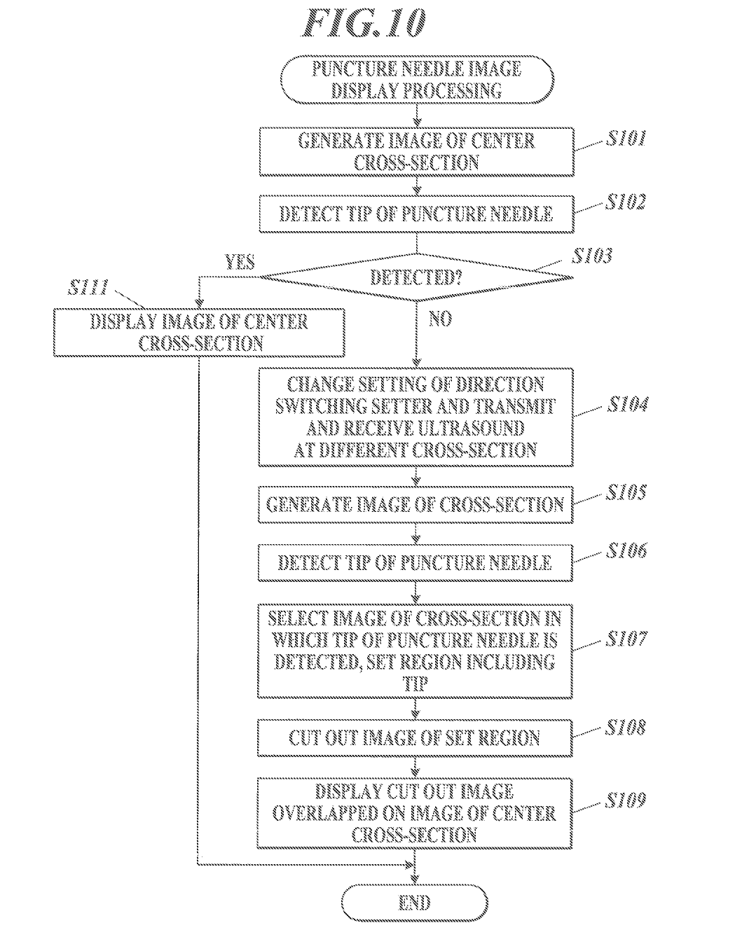

Next, an example of ultrasound image display is described with reference to FIG. 8A to FIG. 8C and FIG. 9A and FIG. 9B. According to the present embodiment, puncturing is performed by a parallel method where the puncturing is parallel to the long axis direction of the ultrasound probe 2.

FIG. 8A is a diagram showing a setting state of the ultrasound probe 2 and the puncture needle 3 in the ultrasound diagnostic apparatus U of the present embodiment. FIG. 8B, FIG. 8C, FIG. 9A, and FIG. 9B are diagrams showing an example of an ultrasound image display of the puncture needle 3 and the tissue in a subject Q (hereinafter referred to as target G) which is the target of puncture.

According to the ultrasound diagnostic apparatus U of the present embodiment, usually, as shown in FIG. 8B, when the target G and the tip 3a of the puncture needle 3 are both detected together in the image of the same cross-section in the subject Q in a B-mode display, the B-mode image of the range is displayed in a display window Wm.

When the tip 3a of the puncture needle 3 is not detected in the cross-section where the target G is detected, the above-described deflection in the short axis direction is performed to generate the diagnostic image in the changed cross-section. When the tip 3a is detected in the changed cross-section, for example, as shown in FIG. 8C, the image of the predetermined region We (detected region) including the detected portion is displayed fitted in the display window Wm. Here, the outer frame is displayed in the region We and the direction of the deflection to discriminate which side of the original cross-sectional image is changed is displayed so that the user is able to easily understand that the image in the region We is a fitted display. Such display for discrimination can be simply a character or an index displayed in or around the region We, or the color of the type of line of the outer frame of the region We can be changed. Alternatively, the display of the region We can be displayed blinking.

As another example of a display when the tip 3a is detected in the diagnostic image in the cross-section changed from the cross-section where the target G is detected, the B-mode image of both cross-sections are displayed side by side as shown in FIG. 9A. Here, on the left side of the display screen of the output display 19, the display window We displaying the diagnostic image of the cross-section including the target G is displayed, and on the right side of the display screen, the display window Ws displaying the diagnostic image of the cross-section including the tip 3a of the puncture needle 3 is displayed. Here, preferably, the two display windows Wc and Ws are displayed side by side with the position in the height direction aligned.

Each display window Wc and Ws displays which cross-section the image shows. Here, “center” is displayed in the display window Wc on the left side to show that the image is the cross-section which is not deflected, and “front” is displayed in the display window Ws on the right side to show that the image is a cross-section of the near side than the display direction of the cross-section of the original image displayed in the display window Wc on the left side.

As a different example of display of the tip 3a detected in the diagnostic image of the cross-section changed from the cross-section where the target G is detected, as shown in FIG. 9B, the B-mode image of the cross-section including the target G is displayed in the display window Wm, and the position of the tip 3a of the puncture needle 3 is detected and identified from the diagnostic image of a different cross-section, and a marker 3m (identification index) is displayed in the position in the display window Wm corresponding to the identified position. In other words, in this case, the image of the tip 3a of the puncture needle 3 detected in the deflected cross-section is not directly displayed, and only the display showing the identified position is shown. In this case also, the display shows which side the image of the cross-section in which the tip 3a of the puncture needle 3 is detected is deflected from the original cross-sectional image. As the identification display, simply a character or index can be displayed in the marker 3m or the color, the type of line, or the thickness of the line of the marker 3m can be changed.

FIG. 10 is a flowchart showing a control process by the controller 11 of the display control processing of the puncture needle 3 executed in the ultrasound diagnostic apparatus U of the present embodiment.

The puncture needle display control processing is performed each time a B-mode image is obtained or ultrasound reception data regarding the diagnostic image is obtained in an operation mode which performs detection and identification of the position of the puncture needle 3.

When the puncture needle image display processing starts, the puncture needle imaging controller 111 of the controller 11 controls the image generator 15 to generate a normal diagnostic image, in other words, the diagnostic image of the central cross-section based on the received ultrasound data (step S101). The puncture needle imaging controller 111 controls a puncture needle identifier 162 to detect the tip 3a of the puncture needle 3 from the diagnostic image (step S102).

The puncture needle imaging controller 111 determines whether the tip 3a of the puncture needle 3 is detected in the diagnostic image of the central cross-section (step S103). When it is judged to be detected (step S103, “YES”), the puncture needle imaging controller 111 displays on the output display 19 the diagnostic image in the central cross-section (step S111). Here, the puncture needle imaging controller 111 is able to display the tip 3a emphasized by displaying the outer frame surrounding the region including the detected tip 3a of the puncture needle 3. Then, the puncture needle imaging controller 111 ends the puncture needle image display processing.

When it is judged that the tip 3a of the puncture needle 3 is not detected (step S103, “NO”), the puncture needle imaging controller 111 outputs a control signal to the direction switching setter 24 to change the setting of the direction switching setter 24, and deflects the transmission and reception direction of the ultrasound to both sides in relation to the central cross-section sequentially to transmit and receive the ultrasound (step S104). With this, the tip 3a of the puncture needle 3 is surveyed. Here, as described above, the puncture needle imaging controller 111 is able to adjust the angle of deflection or, instead of deflection, make the opening width smaller to suppress the convergence of the ultrasound according to the position where the tip 3a of the puncture needle 3 is predicted to be (distance from the ultrasound transmission/reception surface, presumed amount of shifting).

The puncture needle imaging controller 111 controls the image generator 15 to generate the diagnostic image for each deflection direction using the obtained ultrasound reception data in the deflection direction (step S105). The puncture needle imaging controller 111 controls the puncture needle identifier 162 to detect the tip 3a of the puncture needle 3 from the diagnostic image (step S106).

The puncture needle imaging controller 111 selects the image of the cross-section in which the tip 3 of the puncture needle 3 is detected and sets the region including the tip 3a in the selected image (step S107). Then, the puncture needle controller 111 cuts out the image of the set region (step S108).

The puncture needle imaging controller 111 overlaps the cut out image in the position corresponding to the diagnostic image of the central cross-section generated at the beginning and displays the image with the output display 19 (step S109). Here, the puncture needle imaging controller 111 is able to display the outer frame of the cutout image with color or blinking. With this, it is possible to show the side to which the cross-section is deflected in relation to the central cross-section in the cross-section where the tip 3a is detected. Then, the puncture needle imaging controller 111 ends the puncture needle display control processing.

### [Modification 1]

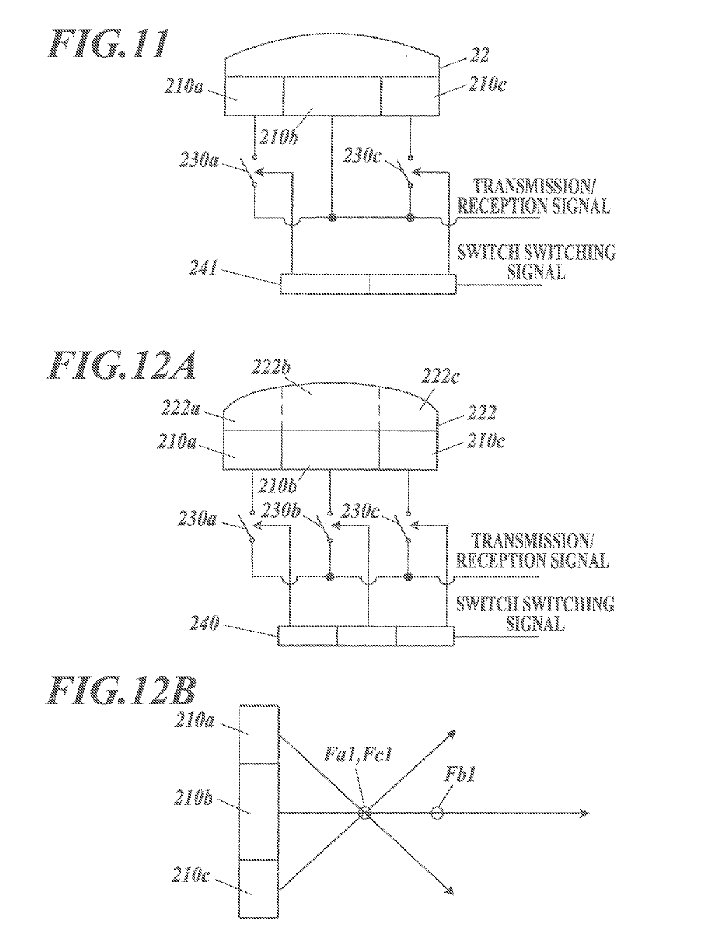

FIG. 11 is a diagram showing a modification 1 of the cross-sectional configuration of the transducer array 21 in the ultrasound probe 2 of the present embodiment along the short axis direction.

According to modification 1, the switching element 230b is not provided for the transducer 210b provided in the center in the short axis direction, and the transducer 210b continuously transmits and receives ultrasound. As described above, if the transducer 210b at the center in the short axis direction is not used in the ultrasound transmission and reception, the transmission/reception strength of the ultrasound drastically decreases. Therefore, when it is difficult to identify the target of image display such as the puncture needle 3 or the target G in the generated diagnostic image, it is possible to reduce the amount of control signals and the size of the register 241 by not providing the switching element 230b from the beginning.

As described above, according to the ultrasound probe 2 provided in the ultrasound diagnostic apparatus U of the first embodiment, the ultrasound probe 2 includes at least, the transducer array 21 which is a plurality of transducers 210 which are arrayed in a short axis direction, and which transmit ultrasound to a subject and receives the reflected wave; the acoustic lens 22 which converges in the short axis direction the ultrasound transmitted and received by the plurality of transducers 210; the switching elements 230a to 230c (230) which turn off the transmission and reception of the ultrasound of some of the plurality of transducers 210a to 210c arrayed in the short axis direction; and the direction switching setter 24 which performs switching operation of turning on and turning off of the switching element 230 to deflect in the short axis direction the transmission and reception direction of the ultrasound by the transducer array of the plurality of transducers 210a to 210c.

As described above, the imaging range can be easily changed by simply controlling the on and off of the switching element 230. Therefore, it is possible to provide an ultrasound probe which has a simple structure and which is easy to use.

According to the ultrasound diagnostic apparatus U of the present embodiment, the ultrasound diagnostic apparatus U includes, the above-described ultrasound probe 2, the transmission driver 12, the reception processor 13, and the transmission/reception switcher 14 which function as the transmission/reception processor to allow the ultrasound probe 2 to perform processing regarding the transmission and reception of the ultrasound, and the controller 11 which outputs the control signal to perform the switching operation of turning on and turning off of the switching element 230 regarding the switching of the deflection direction by the direction switching setter 24.

Therefore, when the user desires to finely adjust the position of the cross-section regarding the B-mode image in the ultrasound diagnostic apparatus U, the cross-section can be easily changed.

The ultrasound diagnostic apparatus U further includes the image generator 15 which generates the diagnostic image regarding the structure of the subject in the cross-section including the transmission/reception direction of the ultrasound and the scanning direction (long axis direction) perpendicular to the transmission/reception direction and the short axis direction based on the received ultrasound; the puncture needle identifier 162 which identifies the tip 3a of the puncture needle 3 in the diagnostic image when the puncture needle 3 is inserted in the subject along the cross-section where the diagnostic image is generated, and the puncture needle imaging controller 111 which outputs to the direction switching setter 24 the control signal to change the deflection direction when the tip 3a is not detected in the diagnostic image, and the puncture needle identifier 162 detects the tip 3a of the puncture needle 3 from the diagnostic image regarding the structure in the cross-section according to the changed deflection direction.

Therefore, when the inserted puncture needle 3 falls out of the imaged cross-section of the diagnostic image, the ultrasound probe 2 with the simple structure can be used to image the cross-section including the tip 3a of the puncture needle 3 with a simple operation so that the tip 3a of the puncture needle 3 can be identified.

The controller 11 functions as the display controller which controls the output display 19 to display the generated diagnostic image. When the tip 3a of the puncture needle 3 is detected from the diagnostic image regarding the structure in the cross-section according to the deflection direction, the detected tip 3a is displayed with the display of the diagnostic image regarding the structure in the cross-section which is not deflected.

Therefore, the user is able to easily understand the relation between the positions of the target G and the puncture needle 3 at the same time with one display even if the puncture needle 3 shifts from the original inserting direction and is outside the imaging range of the original diagnostic image. Therefore, the user is able to easily correct the inserting direction of the puncture needle 3 to be inserted in the correct direction and the process regarding the insertion of the puncture needle 3 becomes easy.

The controller 11 sets the predetermined region We including the detected tip 3a and displays the image of the region We overlapped in the corresponding position on the display window Wm displaying the diagnostic image regarding the structure in the cross-section which is not deflected. Therefore, the user is further able to easily understand the relation between the positions of the target G and the puncture needle 3.

Alternatively, the controller 11 displays the predetermined marker 3m showing the detected tip 3a in the corresponding position on the display window Wm displaying the diagnostic image regarding the structure in the cross-section which is not deflected. Therefore, even if the diagnostic image including the tip 3a has a low gain (S/N ratio) compared to the diagnostic image regarding the cross-section which is not deflected, there is no need to adjust the images, and the position of the tip 3a can be shown easily and clearly regardless of the identification accuracy of the identified tip 3a.

The controller 11 also controls display showing the direction of deflection on the display of the tip 3a. Therefore, the user is able to easily understand which side the puncture needle 3 is shifted from the original cross-section. Consequently, the user is able to easily and promptly correct the inserting direction of the puncture needle 3 to insert the puncture needle 3 in the correct direction.

The puncture needle imaging controller 111 outputs to the direction switching setter 24 the control signal setting the deflection direction differently for each transducer line with a different position in the scanning direction, and adjusts the deflection direction of the ultrasound transmitted and received in one time. Therefore, compared to changing the deflection direction of all of the transducer lines in the same way, the deflection direction can be adjusted more finely, the tip 3a of the puncture needle 3 can be imaged accurately at a high gain in the possible range and the position of the tip 3a of the puncture needle 3 can be identified.

### Second Embodiment

Next, the ultrasound diagnostic apparatus of the second embodiment is described.

The configuration and the operation of the ultrasound diagnostic apparatus U of the second embodiment is the same with the exception of the shape of the ultrasound transmission/reception surface of the ultrasound probe 2 of the ultrasound diagnostic apparatus U of the first embodiment. Therefore, detailed description of the configuration and the operation is omitted.

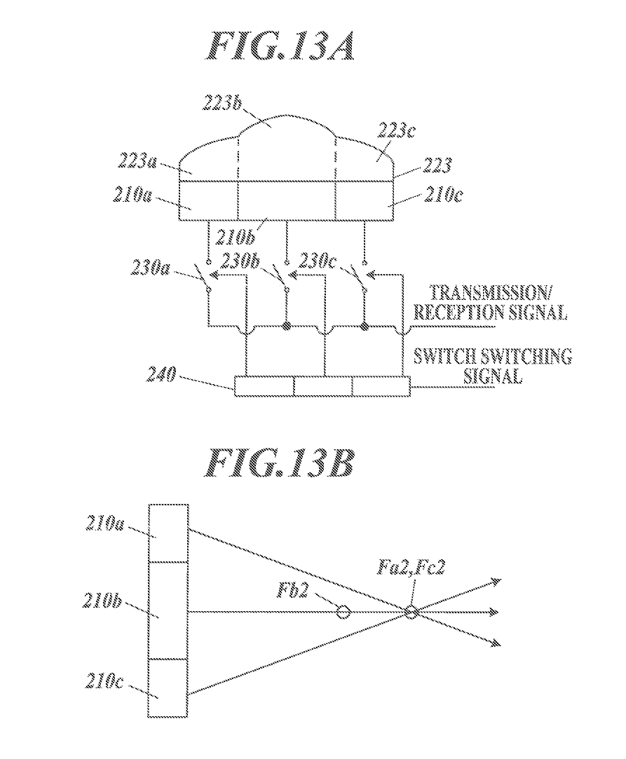

The transmission/reception range of the ultrasound in the ultrasound probe 2 of the second embodiment is described with reference to FIG. 12A and FIG. 12B. FIG. 12A is a diagram showing a cross-sectional configuration regarding the transducer array 21 in the ultrasound probe 2 of the present embodiment along the short axis direction. FIG. 12B is a diagram describing the ultrasound transmission/reception direction of the transducers 210a and 210b.

As shown in FIG. 12A, according to the ultrasound probe 2 of the present embodiment, the curvature of the acoustic lens 222 is different according to the range of the transducers 210a to 210c. Here, the curvature radius of the lens portion 222b for the transducer 210b at the center in the short axis direction is to be larger than the curvature radius of the lens portion 222a and 222c for the transducers 210a and 210c. As a result, as shown in FIG. 12B, the ultrasound focal positions Fa1 and Fc1 for the transducers 210a and 210c are a position different from the ultrasound focal position Fb1 for the transducer 210b. The focal positions Fa1 and Fc1 are in a position closer to the ultrasound transmission/reception surface than the focal position Fb1.

The transmission/reception beam of the ultrasound from the transducer with a predetermined area (width) becomes finest at the focal position. When the distance from the ultrasound transmission/reception surface to the focal positions Fa1 and Fc1 of the transducers 210a and 210c is the same as the distance to the focal position Fb1 of the transducer 210b, the central position of the transmission/reception range does not shift in the short axis direction at the focal position. As a result, when the puncture needle 3 is in the focal position, the tip of the puncture needle 3 is not within the transmission/reception range of the fine ultrasound beam which is not shifted to the short axis direction, and the image of the puncture needle 3 cannot be obtained.

According to the ultrasound diagnostic apparatus U of the present embodiment, the curvature radiuses of the lens portions 222a and 222c for the transducers 210a and 210c are different from the curvature radius of the lens portion 222b for the transducer 210b. With this, the ultrasound focal positions Fa1 and Fc1 are different from the ultrasound focal position Fb1 for the transducer 210b. Here, the focal distance of the lens portions 222a and 222c regarding the transducers 210a and 210c provided in the edge side of the transducer line is made shorter than the focal distance of the lens portion 222b of the transducer 210b provided in the center side of the transducer line. In other words, the curvature radius (refractive index) is set so that the focal positions Fa1 and Fc1 are set to a position closer to the ultrasound transmission/reception surface than the focal position Fb1. With this, the shifting amount of the center of the ultrasound transmission/reception beam is zero in the short axis direction in a position close to the ultrasound transmission surface in which the possibility that the puncture needle 3 is away from the target direction is low, and it is possible to shift in the predetermined direction in the position deep from the ultrasound transmission/reception surface. In the focal positions Fa1 and Fc1, the ultrasound beam regarding the transmission and the reception by the transducer 210b is not fine, and in the focal position Fb1, the ultrasound beam regarding the transmission and reception of the transducers 210a and 210c is not fine. Therefore, the ultrasound reception range regarding generating the diagnostic image can become larger.

### [Modification 2]

The transmission/reception range of the ultrasound in the modification of the ultrasound probe 2 of the second embodiment is described with reference to FIG. 13A and FIG. 13B. FIG. 13A is a diagram showing a cross-sectional configuration of the transducer array 21 in the ultrasound probe 2 of the modification 2 along the short axis direction. FIG. 13B is a diagram describing the ultrasound transmission/reception direction of the transducers 210a and 210b.

According to the ultrasound probe 2 of the present modification, opposite of the acoustic lens 222 of the ultrasound probe 2 of the second embodiment, the lens portions 223a and 223c corresponding to the transducers 210a and 210c of the acoustic lens 223 are formed with a curvature radius larger than the curvature radius of the lens portion 223b corresponding to the transducer 210b. As a result, the focal positions Fa2 and Fc2 of the transmitting and receiving ultrasound regarding the transducers 210a and 210c are farther than the focal position Fb2 of the transmitting and receiving ultrasound regarding the transducer 210b.

As described above, by shifting the focal positions Fa2 and Fc2 to a position farther from the ultrasound transmission/reception surface than the focal position Fb2, when the tip of the puncture needle 3 is shifted in the short axis direction at the focal position, the tip of the puncture needle 3 can be easily included in the diagnostic image. In this case, compared to the setting of the focal positions Fa1 and Fc1 regarding the above-described second embodiment, it is difficult to achieve a large shifting amount to the short axis direction in a position farther from the ultrasound transmission/reception surface than the focal position Fb1.

As described above, according to the ultrasound diagnostic apparatus U of the second embodiment, the acoustic lens 222 is formed for each lens portion 222a to 222c so that the focal positions Fa1 to Fc1 of the ultrasound transmitted and received by the plurality of transducers 210a to 210c arrayed in the width direction are set to a plurality of different positions. Therefore, even if the imaging subject such as the imaging target G and the puncture needle 3 are relatively near the focal point of the acoustic lens 222, the imaging range can be changed easily and accurately and the imaging subject which is originally out of the imaging range can be included in the imaging range. Therefore, the user can understand the position of the imaging subject more reliably regardless of the depth of the imaging target. When the inserting direction of the tip 3a of the puncture needle 3 is shifted, the position can be corrected promptly.

Specifically, the curvature radius (refractive index) of the acoustic lens 222 is set so that, among the plurality of transducers 210a to 210c arrayed in the width direction, the focal distance of the ultrasound transmitted and received by the transducers 210a and 210c provided in the edge side of the transducer array is shorter than the focal distance of the ultrasound transmitted and received by the transducer 210b provided in the center side of the transducer line. With this, when the transmission/reception direction of the ultrasound is deflected, the direction can be largely changed efficiently. Therefore, the percentage of the imaging target such as the tip 3a of the puncture needle 3 not being inside of the imaging range even when the transmission/reception direction of the ultrasound is deflected can be suppressed to a low percentage.

The present invention is not limited to the above embodiments and various changes are possible.

For example, according to the above-described embodiments, the deflection direction is changed according to the position of the tip 3a of the puncture needle 3 when the puncture needle 3 is inserted. However, the ultrasound diagnostic apparatus U and the ultrasound probe 2 of the present invention can be used regardless of whether the puncture needle 3 is being inserted. For example, when a small target G is imaged, a plurality of diagnostic images with different deflection directions can be generated to identify the target G automatically or by input by the user. From then on, the deflection direction that the target G is imaged most clearly can be fixed and real time imaging can be performed to lighten the burden of fine adjustment by the user.

According to the present embodiment, when the transmission/reception direction of the ultrasound is deflected, a coefficient multiplied according to the distance from the ultrasound transmission/reception surface to correct the reduction of the reception sensitivity (S/N ratio) is stored in the storage of the controller 11. However, a function can be held and the calculation can be executed each time. When the change according to the distance based on the setting of the various parameters is small, simply a fixed number can be multiplied regardless of the distance.

According to the present embodiment, the control operation to set the direction switching setter 24 is performed in the ultrasound diagnostic apparatus main body 1. Alternatively, the direction switching setter 24 can include a controller (switching controller) and the control operation regarding the switching of the switching elements 230a to 230c can be performed in the ultrasound probe 2. Instead of the input operation to the operation inputter 28 of the ultrasound probe 2 or in addition to such input operation, setting such as switching the deflection direction according to input operation on the operation inputter 18 of the ultrasound diagnostic apparatus main body 1 can be performed.

With this, the switching operation regarding the deflection of the imaging range can be completed inside the ultrasound probe 2. Therefore, the exchange of the control signal with the ultrasound diagnostic apparatus main body 1 becomes easier.

Specifically, when the operation inputter 28 is provided and the switching control is performed based on the input operation from the operation inputter 28, there is no need to provide a signal line regarding control with the ultrasound diagnostic apparatus main body 1. Therefore, the cable 5 can be made smaller and lighter. Moreover, there is no need to reach for and operate the ultrasound diagnostic apparatus main body 1, and therefore, the handling of the ultrasound probe 2 becomes easy and reliable.

Specifically, the ultrasound diagnostic apparatus main body 1 includes the image generator 15 which generates the diagnostic image regarding the structure of the subject in the cross-section including the transmission/reception direction of the ultrasound and the scanning direction (long axis direction) perpendicular to the transmission/reception direction and the width direction based on the received ultrasound, and the puncture needle identifier 162 which identifies the tip 3a of the puncture needle 3 in the diagnostic image when the puncture needle 3 is inserted inside the subject along the cross-section. The ultrasound diagnostic apparatus main body 1 is connected to the ultrasound probe 2 including the direction switching setter 24 including the above-described controller. Consequently, the user is able to easily and reliably handle the ultrasound probe 2 when the puncture needle 3 is inserted.

In the above-described embodiments, the operation inputter 28 is provided in the ultrasound probe 2 to enhance operability. On the other hand, it is possible to not provide the operation inputter 28 and perform all operation on the ultrasound diagnostic apparatus main body 1. With this, careless error in operation can be prevented and the ultrasound probe 2 can be made lighter.

According to the present embodiment, various display is performed on the display screen of the output display 19 provided in the ultrasound diagnostic apparatus main body 1. However, the display can be performed on the display screen of an external device attached through an output interface, or some or all of the information regarding the imaging data can be digitized and output externally.

According to the present embodiment, the ultrasound diagnostic apparatus U includes the ultrasound probe 2 and the ultrasound diagnostic apparatus main body 1. However, the ultrasound probe 2 which can independently perform operation and deflection control can be connected to and used on the normal ultrasound diagnostic apparatus main body.

Alternatively, the ultrasound diagnostic apparatus main body 1 does not need to have the above-described function regarding the display of the tip 3a of the puncture needle 3. In this case, for example, in the ultrasound diagnostic apparatus U, simply the image of the cross-section including the target G and the image of the cross-section including the tip 3a can be generated and displayed alternately.

According to the above-described embodiments, the number of transducers arrayed in the short axis direction is 3. However, the number can be a different number no less than 2. In this case, when the deflection is performed, the combination of the transducer is suitably set so that the ultrasound is not transmitted and received according to the deflection direction and the reception ultrasound strength.

The acoustic lens 22 can be formed as one with the transducers 210 of the transducer array 21, or can be formed for each transducer 210 in the same position in the long axis direction or the short axis direction, or can be formed separately for all of the transducers 210.

According to the present embodiment, the scanning direction of the ultrasound reception is set as the long axis, however, the number of lines can be suitably provided regardless of the number of array of the transducer 210 in the short axis direction according to the length of the scanning direction and necessity of scanning.

The array in the scanning direction does not have to be a linear scanning type, and other array is possible, for example, a sector scanning type, a convex type, a radial scanning type and the like.

According to the above-described embodiment, the puncture needle 3 is part of the ultrasound diagnostic apparatus U attached to the ultrasound probe 2 with the attachment 4. Alternatively, the puncture needle can be a configuration separate from the ultrasound diagnostic apparatus U as long as the puncture needle is inserted while displaying the diagnostic image.

The present invention is not limited to the embodiments and configurations as described above, and the embodiments can be suitably modified without leaving the scope of the invention.

The present U.S. patent application claims priority under the Paris Convention of Japanese Patent Application No. 2014-173730 filed on Aug. 28, 2014 the entirety of which is incorporated herein by reference.

What is claimed is:

1. An ultrasound probe comprising:
a plurality of transducers which are arrayed in at least a predetermined first direction and which transmit ultrasound to a subject to receive a reflected wave;
a convergence unit which converges the ultrasound transmitted and received by the plurality of transducers to the first direction;
a switching element which turns off transmission and reception of the ultrasound of some of the plurality of transducers arrayed in the first direction; and
a direction switching setter which performs switching operation of turning on and turning off of the switching element to deflect in the first direction a transmission/reception direction of the ultrasound by the plurality of transducers.

2. The ultrasound probe according to claim 1, wherein, the convergence unit is formed so that focal positions of the ultrasound transmitted and received by the plurality of transducers arrayed in the first direction are set in a plurality of different positions.

3. The ultrasound probe according to claim 2, wherein, the convergence unit is set so that a focal distance of the ultrasound transmitted and received by the transducer provided in an edge side of the array of the plurality of transducers arrayed in the first direction is shorter than a focal distance of the ultrasound transmitted and received by the transducer provided in a center side of the array.

4. The ultrasound probe according to claim 1, further comprising:
a switching controller which outputs a control signal to the direction switching setter,
wherein, the direction switching setter performs the switching operation of the switching element based on the control signal.

5. The ultrasound probe according to claim 4, further comprising:
an inputter which receives input operation by a user,
wherein, the switching controller outputs the control signal according to input operation on the inputter.

6. The ultrasound probe according to claim 4, wherein, the switching controller outputs the control signal set so that the direction of deflection is different for each array of transducers in the first direction in a different position in a second direction perpendicular to the first direction and the transmission/reception direction of the ultrasound.

7. An ultrasound diagnostic apparatus comprising:
the ultrasound probe according to claim 1;
a transmission/reception processor which controls the ultrasound probe to transmit and receive the ultrasound; and
a switching controller which outputs a control signal to the direction switching setter,
wherein, the direction switching setter performs the switching operation of the switching element based on the control signal.

8. An ultrasound diagnostic apparatus comprising:
the ultrasound probe according to claim 4; and
a transmission/reception processor which controls the ultrasound probe to transmit and receive the ultrasound.

9. The ultrasound diagnostic apparatus according to claim 7, further comprising:
an image generator which generates a diagnostic image regarding a structure of a subject in a cross-section including a transmission/reception direction of the ultrasound and a second direction perpendicular to the transmission/reception direction and the first direction based on the received ultrasound; and
a puncture needle identifier which identifies a tip portion of the puncture needle in the diagnostic image when a puncture needle is inserted inside the subject along the cross-section,
wherein, the switching controller outputs the control signal which changes a direction of the deflection when the tip portion is not detected in the diagnostic image; and
the puncture needle identifier detects the tip portion of the puncture needle from the diagnostic image regarding the structure in the cross-section according to the changed direction of deflection.

10. The ultrasound diagnostic apparatus according to claim 9, further comprising:
a display controller which displays the generated diagnostic image on the display,
wherein, the display controller displays the diagnostic image regarding the structure in the cross-section which is not deflected with the detected tip portion when the tip portion of the puncture needle is detected from the diagnostic image regarding the structure in the cross-section according to the deflected direction.

11. The ultrasound diagnostic apparatus according to claim 10, wherein, the display controller sets a predetermined detected region including the detected tip portion and displays an image of the detected region overlapped on a corresponding position in the diagnostic image regarding the structure in the cross-section which is not deflected.

12. The ultrasound diagnostic apparatus according to claim 10, wherein, the display controller displays a predetermined identification index showing the detected tip portion in a corresponding position on the diagnostic image regarding the structure in the cross-section which is not deflected.

13. The ultrasound diagnostic apparatus according to claim 10, wherein, the display controller displays a direction of the deflection together with the display of the tip portion.

14. The ultrasound diagnostic apparatus according to claim 9, wherein, the switching controller outputs the control signal set so that a direction of the deflection is different for each array of the transducers in the first direction in a position different in the second direction, and the direction of the deflection of the ultrasound transmitted and received at one time is adjusted.
